(19) United States
(12) Patent Application Publication
(10) Pub. No.: US 2012/0021064 A1
(43) Pub. Date: Jan. 26, 2012

(54) COMPOSITIONS FOR BOWEL PREPARATION AND METHODS OF USE THEREOF

(75) Inventor: Stephana Patton, San Francisco, CA (US)

(73) Assignee: Salix Pharmaceuticals, Ltd., Raleigh, NC (US)

(21) Appl. No.: 13/246,287

(22) Filed: Sep. 27, 2011

Related U.S. Application Data
(63) Continuation of application No. PCT/US2010/028962, filed on Mar. 26, 2010.
(60) Provisional application No. 61/164,381, filed on Mar. 27, 2009.

(51) Int. Cl. A61K 33/42; A61P 1/00; G01N 33/48; C12Q 1/02

(52) U.S. Cl. 424/606; 435/29; 73/61.43

(57) ABSTRACT

The present invention relates to formulations and kits for gastrointestinal cleansing and to therapeutic methods thereof.

### RELATED APPLICATIONS

This application is a continuation of International Application No. PCT/US2010/028962, filed Mar. 26, 2010, which claims benefit of U.S. Provisional Application No. 61/164,381, filed Mar. 27, 2009. The entire contents of each of the aforementioned applications are expressly incorporated herein by reference.

### BACKGROUND

In an attempt to avoid the problems associated with the high-volume gastro-intestinal cleansing preparations, smaller-volume aqueous preparations comprising of phosphate salts have been marketed. The phosphate salt solution produces an osmotic effect, causing large amounts of water to be drawn into the bowel, thereby promoting bowel evacuation. Although the lower volume marginally favors these sodium phosphate preparations, adverse side effects such as nausea, vomiting (principally a result of unpalatable taste), abdominal bloating, pain and dizziness were of similar frequency compared to polyethylene glycol-electrolyte lavage (Kolts et al. (1993) Am. J. Gastroenterol. 88:1218-1223). Oral tablets containing phosphate salts have been formulated (see U.S. Pat. Nos. 5,616,346 and 6,162,464) to increase preparatory compliance, reduce volume discomfort, and increase patient tolerance. The oral tablet formulation significantly reduced the incidence of gastrointestinal adverse events such as nausea, vomiting, and bloating (Rex et al. (2002) Aliment Pharmacol. Ther. 16:937-944). Further, these tablet formulations were significantly better accepted and preferred by patients. There is need for methods of selecting subject who should not take certain colonic purgative compositions because of adverse side effects.

### SUMMARY

Presented herein, according to one aspect are methods of cleansing the GI tract of a subject, comprising administering about 48 mg of sodium phosphate with about 2 L of aqueous solution to a subject who has not recently had a kidney biopsy indicating kidney damage due to excess phosphate.

Presented herein, according to one aspect are methods of cleansing the GI tract of a subject, comprising determining weather a subject is a candidate for a sodium phosphate GI cleanser; providing about 48 mg of sodium phosphate to a subject; and instructing the subject to ingest the sodium phosphate with 2 L of aqueous solution.

According to one embodiment, determining comprises a biopsy of the kidney, wherein if the biopsy shows damage to the kidney due to too much phosphate, the subject is not a candidate for a sodium phosphate GI cleanser.

Presented herein, according to one aspect are methods of determining if a subject is a candidate for cleansing the GI tract with sodium phosphate comprising determining the phosphate levels in a kidney of the subject; wherein normal levels of phosphate in the kidney indicates that the subject is a candidate for cleansing of the GI tract with sodium phosphate.

According to one embodiment, a biopsy is performed to obtain a sample of the kidney from the subject.

In one embodiment, the methods further comprise administering to a subject determined to be a candidate about 48 mg of sodium phosphate with about 2 L of aqueous solution.

Presented herein, according to one aspect are methods of determining if a subject is a candidate for cleansing the GI tract with sodium phosphate comprising determining if the subject has normal renal function; wherein normal renal function indicates that the subject is a candidate for cleansing of the GI tract with sodium phosphate.

According to one embodiment, normal renal function is characterized by creatinine clearance of greater than or equal to 30 mL/minute.

In one embodiment, the methods further comprise administering to a subject determined to be a candidate about 48 mg of sodium phosphate with about 2 L of aqueous solution.

Presented herein, according to one aspect are methods of determining if a subject is a candidate for cleansing the GI tract with sodium phosphate comprising determining if the subject is a candidate for sodium phosphate purgative cleansing by one or more of: determining age, determining renal function, or determining phosphate level of the kidney; and administering to a subject determined to be a candidate about 48 mg of sodium phosphate with about 2 L of aqueous solution.

According to one embodiment, if a subject is determined to be of increased age, they are not a candidate.

According to one embodiment, determined to have abnormal levels of phosphate in the kidney they are not a candidate.

According to one embodiment, if a subject has renal function characterized by creatinine clearance of greater than or equal to 30 mL/minute they are a candidate.

Other embodiments of the invention are disclosed infra.

### DETAILED DESCRIPTION

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Administration “in combination with” one or more further therapeutic agents includes simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved.

As used herein, an “increase” or “decrease” in a measurement, unless otherwise specified, is typically in comparison to a baseline value. For example, an increase in time to hospitalization for subjects undergoing treatment may be in comparison to a baseline value of time to hospitalization for subjects that are not undergoing such treatment. In some instances an increase or decrease in a measurement can be evaluated based on the context in which the term is used.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

The term “effective amount” includes an amount effective, at dosages and for periods of time necessary, to achieve the desired result, e.g., an amount of sodium phosphate sufficient to cleanse the GI tract in a patient or subject. For example, an effective amount is about 48 mg of sodium phosphate with about 2 L of aqueous solution. Dosage regimens may be adjusted to provide the optimum response. An effective amount is also one in which any toxic or detrimental effects (e.g., side effects) of a GI specific antibiotic are outweighed by the therapeutically beneficial effects.

Any binder that is soluble, or soluble and nonfermentable, may be used in the sodium phosphate GI cleanser formulation. However, binders that are fermentable, like any other fermentable ingredient, should only be used in embodiments where a spark would not be produced in the colon. A soluble, nonfermentable binder that may be used in the formulations of the invention includes, but is not limited to, polyethylene glycol (PEG). Applicants discovered that a purgative composition containing the soluble, nonfermentable binder PEG, leaves little or no residue after use for bowel preparation, thereby increasing visualization of the colon. PEG is represented by the structural formula: HOCH₂(CH₂OCH₂)ₘCH₂OH, wherein m represents the average number of oxyethylene groups.

Any PEG polymer may be employed in the compositions contemplated herein. In one embodiment, the PEG polymers are solid at room temperature (i.e., 25° C.) and/or soluble in (or miscible with) water at room temperature. In one embodiment, the average molecular weight of the PEG polymer is at least 200, at least 400, at least 600, at least 1,000, at least 1540, at least 3000, at least 4,000, or at least 8,000. In one embodiment, the average molecular weight of the PEG polymer is from 7,000 to 9,000.

The amount of soluble and/or non-fermentable binder may vary depending on the desired characteristics of the solid dosage form and can be determined by one of ordinary skill in the art. In one embodiment, a PEG binder comprises 5-20%, in another embodiment 7.5-15%, and in an additional embodiment 10% by weight.

In one embodiment, the composition of the invention is free of insoluble binder or only contains levels of insoluble binder that do not impede the visualization of the colon.

Various purgatives are available commercially, and any available form of the material can be used in the practice of this invention. Purgatives that may be used in the invention include, but are not limited to, non-osmotic, osmotic, and bulk-forming purgatives. The invention may contain one purgative, more than one purgative from the same category, or more than one purgative from different categories may be used. Many purgatives may have more than one role or function, or may be classified in more than one group. Such classifications are descriptive only, and not intended to limit any use of a particular purgative.

In one embodiment, at least one osmotic purgative is used in the formulation of the invention. Osmotic purgatives act by increasing intestinal osmotic pressure thereby promoting retention of fluid within the bowel. Osmotic purgatives that may be included in the composition include salts, for example, magnesium citrate, magnesium chloride, magnesium hydroxide, magnesium phosphate, magnesium sulfate, magnesium tartrate, sodium phosphate, sodium tartrate, sodium sulfate, potassium tartrate, magnesium oxide, sodium sulfate, or salts thereof. Other examples of osmotic purgatives include glycerin, sorbitol, mannitol, lactitol, alcohol sugars, L-sugars (e.g., L-glucose), polyethylene glycol, and lactulose.

In one embodiment, the at least one purgative is sodium phosphate or a salt thereof. In an additional embodiment of the invention, the at least one purgative is monobasic sodium phosphate, dibasic sodium phosphate, or tribasic sodium phosphate.

Salts according to the sodium phosphate GI cleanser formulation may be used in a variety of forms, for example anhydrous or a hydrated form. It is also contemplated that a change in the form of a salt may increase or decrease its molecular weight. To account for any change in molecular weight, components of the purgative formulation and/or amounts of the purgative salts may be adjusted according to the knowledge of the person of ordinary skill in the art. In one embodiment, monobasic sodium phosphate is used in a monohydrate form. In another embodiment of the invention, dibasic sodium phosphate is used in an anhydrous form.

In one embodiment, the formulation of the invention comprises at least one non-osmotic purgative. Non-osmotic purgatives include prokinetic laxatives that stimulate the motility of the gastrointestinal tract, as well as stimulant laxatives that act by directly stimulating nerve endings in the colonic mucosa. Emollient laxatives and mucosal protectants may also be used in the invention. Examples of non-osmotic purgatives that may be used in the invention include, but are not limited to, mineral oil, aloe, bisacodyl, sodium picosulfate, casanthranol, cascara, castor oil, danthron, dehydrocholic acid, phenolphthalein, sennosides, docusate, bethanachol, colchicines, misoprostol, cisapride, norcisapride, paraffin, rhein, and tegaserod.

In one embodiment, the colonic purgative composition contains at least one osmotic purgative and at least one non-osmotic purgative.

In addition to at least one osmotic purgative and/or at least one non-osmotic purgative, the colonic purgative formulations of the invention may also comprise at least one bulk-forming purgative. Bulk-forming purgatives cause retention of fluid and an increase in fecal mass, resulting in stimulation of peristalsis. Bulk-forming laxatives may include various natural and semisynthetic polysaccharides, cellulose derivatives, or other substances that dissolve or swell in water to form an emollient gel or viscous solution that serves to maintain the feces soft and hydrated. Examples of bulk-forming purgatives that can be used in the invention include, but are not limited to, methylcellulose, sodium carboxymethyl cellulose, bran, psyllium, sterculia, and testa ispaghula.

Additional Optional Ingredients

Additional optional components may be included in the formulations of this invention to, for example, enhance the characteristics of the solid dosage form, maintain the integrity of particles of the active ingredient during the formulation process, and/or enhance the safety of the formulation. Any additional components may be compatible with the other ingredients in the formulations of the invention, in particular the active ingredients, and may not adversely affect the osmolarity of the formulations. Additional optional ingredients that may be used in the formulations of the invention include, for example, coatings, diluents, binders, glidants, lubricants, colors, disintegrants, flavors, sweeteners, polymers or waxes.

Lubricants, for example, may be included in the formulations of the invention. Such lubricants include, but are not limited to, magnesium stearate, potassium stearate, talc, stearic acid, sodium lauryl sulphate, and paraffin. In one embodiment, the colonic purgative formulation further comprises magnesium stearate. Lubricants serve to facilitate the manufacturing of a solid dosage form.

Additional suitable ingredients also include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethyl-cellulose, alginates, gelatin, polyvinyl pyrrolidone, sucrose, and acacia; humectants, such as glycerol; disintegrating agents, such as agar, calcium carbonate, potato and tapioca starch, alginic acid, certain silicates, colloidal silicon dioxide, sodium starch glycolate, crospovidone, and sodium carbonate; solution retarding agents, such as paraffin; absorption accelerators, such as quaternary ammonium compounds; wetting agents, such as cetyl alcohol and glycerol monostearate; absorbents, such as kaolin and bentonite clay; stabilizers, such as fumaric acid; coloring agents; buffering agents; dispersing agents; preservatives; organic acids; and organic bases.

In one embodiment, an additional component in the formulations of the invention may function to maintain the electrolyte balance in a patient. For example, formulations of the invention may further comprise calcium, phosphate, potassium, magnesium, other anions, or salts thereof, which may normally be lost in diarrhea fluid.

Acidic or basic compounds may also be optionally added to the composition to adjust the pH of the compound or to alter the disintegration characteristics. Acidic or basic compounds that may be included in the formulations of the invention include, but are not limited to, sodium carbonate, sodium bicarbonate, sodium phosphate, calcium carbonate, magnesium hydroxide, potassium hydroxide, magnesium carbonate, and aluminum hydroxide.

The aforementioned ingredients are given as examples only and are not meant to include all possible choices. Additionally, many may have more than one role or function, or be classified in more than one group. Such classifications are descriptive only, and not intended to limit any use of a particular component.

To optimize the solid dosage formulations, components and amounts of the colonic purgative formulations of the invention may be adjusted according to the knowledge of the person of ordinary skill in the art. Not all of the components are necessary, but are provided for illustration only. For example, it may not be necessary to have two distinct purgatives and it may also not be necessary to have a lubricant, such as magnesium stearate.

GI cleansers, as used herein include purgatives and constipation relievers, which are also known as, oral laxative solutions (e.g., laxative preparations), colon clearing composition, bowel irrigation, enemas, rectal pulsed irrigation and bowel preparations. As used herein refer to compounds or compositions that free the bowel from solid matter (e.g., stool). Combinations of GI cleansers and other stimulation compositions may be useful, for example, use of a stimulant laxative (e.g., bisacodyl) in combination with an osmotic laxative. The GI cleanser may be one or more of a sodium phosphate based composition or a sodium phosphate composition in combination with a PEG based composition as further described below. GI cleansers may also be combinations of the below described cleansers or other cleansers known by one of skill in the art to be effective according to the methods described herein.

Exemplary stimulant laxatives include, for example, Aloe, 250-1000 mg; Bisacodyl, about 5-80 mg; Casanthranol, 30 to 360 mg; Cascara aromatic fluid extract, 2-24 ml.; Cascara sagrada bark, 300-4000 mg; Cascada sagrada extract, 300 to 2000 mg; Cascara sagrada fliuid extract, 0.5 to 5 ml.; Castor oil, 15-240 ml.; Danthron, 75-300 mg; Dehydrocholic Acid, 250-2000 mg; Phenolphthalein, 30-1000 mg; Sennosides A and B, 12-200 mg; and Picosulfate, 1-100 mg. Of course, larger or smaller doses may be used, as necessary, to produce a bowel movement within less than about 12 hours, while avoiding unnecessary discomfort.

Bisacodyl is a stimulant laxative, available without prescription, used to treat constipation. Bisacodyl is available in tablets, suppositories, and in premixed enema formulations. Bisacodyl enemas are usually effective to produce a bowel movement in about 20 minutes, suppositories usually produce a bowel movement in about an hour, and oral administration of a tablet usually results in a bowel movement in about 3 to 6 hours.

Polyethylene Glycol (PEG) based solutions 3350 has been used alone as a medication to treat constipation by improving bowel motility, stool formation, or both and comprise, for example, polyethylene glycol (PEG), sodium sulfate, sodium chloride, potassium chloride, ascorbic acid; or PEG, sodium sulfate, sodium chloride, potassium chloride, ascorbic acid, and sodium ascorbate; or PEG 3350, sodium sulfate, sodium chloride, potassium chloride, ascorbic acid, and sodium ascorbate. In one embodiment, the PEG purgative is supplied as two pouch A's comprising 100 grams of polyethylene glycol (PEG) 3350, 7.5 grams of sodium sulfate, 2.691 grams of sodium chloride, and 1.015 grams of potassium chloride; and two pouch B's comprising 4.7 grams of ascorbic acid, and 5.9 grams of sodium ascorbate. It is well know by one of skill in the art how to administer such compositions to produce cleansing.

In certain embodiments, a sodium phosphate GI cleanser useful in the methods described herein comprises 32 or 40 tablets comprising sodium phosphate monobasic, sodium phosphate dibasic, PEG 8000, and magnesium sterate. Another example comprises sodium phosphate monobasic, sodium phosphate dibasic, microcrystalline cellulose, colodial silicon dioxide, and magnesium sterate. Other useful GI cleansers include, for example, Fleet® Phospho-soda® EZ-Prep™; miraLAX; a bulk producing purgative; a serotonin agonist; a hyperosmotic agent; GoLytely; GlycoLax; CoLyte; or NuLytely. One of skill in the art would know how to administer each of these compositions.

Other GI cleansers useful in the methods and formulations (e.g., kits) described herein, include, for example, those described by Fordtran et al. (WO87/00754), including the reduced sodium sulphate solution (RSS). This solution comprises no sodium sulphate but instead has a relatively high concentration of polyethylene glycol (75 to 300 g/l). A solution disclosed in WO87/00754 comprises PEG 3350 (120 g/l), sodium bicarbonate (1.68 g/l), potassium chloride (0.74 g/l) and sodium chloride (1.46 g/l) and it is also administered in a quantity of 4 litres. Another exemplary solution is commercialized by Braintree Laboratories Inc (Braintree, Mass., U.S.A.) under the name NuLYTELY (initially also under the name GoLYTELY-RSS). The NuLYTELY composition comprises PEG 3350 (105 g/l), sodium bicarbonate (1.43 g/l), potassium chloride (0.37 g/l) and sodium chloride (2.80 g/l) and it is supplied in dry powder form for making up to 4 liters. WO 89/05659 (Borody) describes yet another exemplary GI cleanser useful in the methods and formulations described herein. This is an orthostatic lavage solution comprising polyethylene glycol, electrolytes and from 0.25 to 50 g/l ascorbic acid (vitamin C) or a salt thereof. The presence of ascorbic acid or a salt thereof is said to reduce the required volume of solution to 3 liters or less. Whilst about 3 g of ascorbic acid may be absorbed in the intestine (Hornig, D. et al., Int. J. Vit. Nutr. Res., 1980, 50, 309) any further ascorbic acid is reported in WO 89/05659 to contribute to the diarrhea and to inhibit bacterial gas generation and bacterial reproduction. The ascorbic acid is also said to facilitate ingestion of the lavage solution because its pleasant acidic taste masks the usual nauseating taste of the salty polyethylene glycol solution.

Other GI cleansers useful in the methods and formulations described herein, include, for example, Fleet® Phospho-soda® EZ-Prep™ Bowel Cleansing System contains: 1. Two Bottles of Fleet® Phospho-soda® Oral Saline Laxative, Unflavored—Dose 1, 45 mL (1.5 fl.oz.) and Dose 2, 30 mL (1.0 fl.oz.). Net contents 75 mL (2.5 fl.oz.). Active ingredients: each 15 mL contains monobasic sodium phosphate monohydrate 7.2 g and dibasic sodium phosphate heptahydrate 2.7 g; 2. Two Lemonade Flavor Packets net contents 0.07 oz. each; and 3. One 12 fl.oz. mixing cup. Fleet® Phospho-soda® Composition comprises, for example, 15 mL of unflavored Fleet® Phospho-soda® oral saline laxative, which contains 7.2 g monobasic sodium phosphate monohydrate and 2.7 g dibasic sodium phosphate heptahydrate in a stable, aqueous solution. Each 1.5 fl. oz. bottle (45 mL) of Fleet® Phospho-soda® oral saline laxative contains 5004 mg sodium. Each 1.0 fl. oz. bottle (30 mL) of Fleet® Phospho-soda® oral saline laxative contains 3336 mg sodium. Fleet® Phospho-soda®oral saline laxative is sugar-free. Elemental and electrolytic content: mEq Phosphate (PO4) per 15 mL-186.75; mEq Sodium (Na) per 15 mL-72.30; mg Sodium (Na) per 15 mL-1668; mmole Phosphorus (P) per 15 mL-62.25.

OsmoPrep, another useful GI cleanser, comprises 48 grams of sodium phosphate (32 tablets), induces diarrhea, which effectively cleanses the entire colon. Each administration has a purgative effect for approximately 1 to 3 hours. The primary mode of action is thought to be through the osmotic effect of sodium, causing large amounts of water to be drawn into the colon, promoting evacuation. Each OsmoPrep tablet contains 1.102 grams of sodium phosphate monobasic monohydrate, USP and 0.398 grams of sodium phosphate dibasic anhydrous, USP for a total of 1.5 grams of sodium phosphate per tablet. Inert ingredients include polyethylene glycol 8000, NF; and magnesium stearate, NF. OsmoPrep is gluten-free. The recommended dose of OsmoPrep Tablets for colon cleansing for adult patients is 32 tablets (48 grams of sodium phosphate) taken orally with a total of 2 quarts of clear liquids in the following manner: the evening before the colonoscopy procedure: Take 4 OsmoPrep Tablets with 8 ounces of clear liquids every 15 minutes for a total of 20 tablets. On the day of the colonoscopy procedure: Starting 3-5 hours before the procedure, take 4 OsmoPrep Tablets with 8 ounces of clear liquids every 15 minutes for a total of 12 tablets. Patients should be advised of the importance of taking the recommended fluid regimen. It is recommended that patients receiving OsmoPrep be advised to adequately hydrate before, during, and after the use of OsmoPrep. Patients should not use OsmoPrep for colon cleansing within seven days of previous administration. No additional enema or laxative is required, and patients should be advised NOT to take additional agents, particularly those containing sodium phosphate.

Visicol® (sodium phosphate monobasic monohydrate, USP, and sodium phosphate dibasic anhydrous, USP) is a purgative used to clean the colon prior to colonoscopy. Visicol® Tablets are white to off-white compressed tablets, with a monogram “I” on each side of the upper surface and a plain lower surface. Each tablet contains 1.102 grams of sodium phosphate monobasic monohydrate, USP and 0.398 grams of sodium phosphate dibasic anhydrous, USP for a total of 1.5 grams of sodium phosphate per tablet. Inert ingredients include microcrystalline cellulose (MCC), NF; magnesium stearate, NF; and colloidal silicon dioxide, NF. Visicol® is gluten-free. Visicol® tablets, taken in two doses of 30 grams (the complete regimen contains a total of 60 grams of sodium phosphate) approximately twelve hours apart, induces diarrhea, which effectively cleanses the entire colon. Each administration has a purgative effect for approximately 1 to 3 hours. The primary mode of action is thought to be through osmotic action of sodium, causing large amounts of water to be drawn into the colon, promoting colon evacuation. The recommended dose of Visicol® Tablets for colon cleansing for adult patients is 40 tablets (60 grams of sodium phosphate) taken orally with a total of 3.6 quarts of clear liquids in the following manner:

The evening before the colonoscopy procedure: Take 3 Visicol® Tablets (the last dose will be 2 Visicol® Tablets) with 8 ounces of clear liquids every 15 minutes for a total of 20 tablets. On the day of the colonoscopy procedure: Starting 3-5 hours before the procedure, take 3 Visicol® Tablets (the last dose will be 2 Visicol® Tablets) with 8 ounces of clear liquids every 15 minutes for a total of 20 tablets. It is recommended that patients receiving Visicol® be advised to adequately hydrate before, during, and after the use of Visicol®. Patients should not use Visico® within seven days of previous administration. No additional enema or laxative is required, and patients should be advised not to take additional agents, particularly those containing sodium phosphate.

Other exemplary GI cleansers include those detailed in Tables 1 and 2:

TABLE 1

| | Grams/ | |
|---|---|---|
| | Tablet | % by weight |
| Sodium Phosphate Salt: | | |
| Monobasic | 1.102 | 65.752 |
| Dibasic | 0.398 | 23.747 |
| Inert: | | |
| PEG 8000, NF | 0.1676 | 10.000 |
| Magnesium Sterate, | 0.0084 | 0.502 |
| NF | | |
| Total | 1.6760 | 100.001 |

TABLE 2

| | | % by |
|---|---|---|
| Ingredients | Grams/tablet | wt |
| Sodium Phosphate | | |
| Salts: | | |
| Monobasic | 1.102 | 62.436 |
| Dibasic | 0.398 | 22.550 |
| Inert ingredients: | | |
| microcrystalline | 0.22950 | 13.003 |
| cellulose | | |
| magnesium stearate | 0.02645 | 1.499 |
| colodial silicone | 0.00885 | .501 |
| dioxide | | |
| | 1.765 | 99.989 |

GI cleansers that are provided as dry powders or concentrated liquids may be, for example, stirred and dissolve in any a beverage (cold, hot or room temperature) and then drank). GI cleansers provided as liquids may just be drunk.

### Methods of Treatment

The present invention also encompasses methods of using the colonic purgative formulations, e.g., to clean the colon prior to colonoscopy. The colonic purgative formulations of the invention produce a broad range of activities, depending on the dosage administered. The present invention encompasses methods of cleansing the GI tract comprising administering to at least one patient a GI cleanser formulation and allowing said formulation to cleanse the GI tract.

Thus, the present invention also encompasses methods of maintaining the elimination or increasing the elimination of feces in the bowel, comprising administering to at least one patient a colonic purgative formulation and promoting the elimination of feces in the bowel.

In one embodiment, the instant invention provides methods of cleansing the GI tract of a subject by administering to the subject an effective amount of, for example, sodium phosphate and a volume of an aqueous solution. In one embodiment, the subject is administered 1, 2, or 3 liters of aqueous solution with the sodium phosphate.

The instant invention provides methods of determining if a subject is a candidate for cleansing of the GI tract with a colonic purgative such as, for example, sodium phosphate such as Osmoprep. In specific embodiments, the instant invention provides methods for determining the phosphate levels in a kidney of the subject, wherein normal levels of phosphate in the kidney indicates that the subject is a candidate for cleansing of the GI tract with sodium phosphate. The levels of phosphate in a kidney can be determined by taking a biopsy of a subject's kidney and determining the level of phosphate in the biopsy sample.

In another embodiment, the invention provides methods for determining if a subject is a candidate for cleansing the GI tract with sodium phosphate by determining if the subject has normal renal function, wherein normal renal function indicates that the subject is a candidate for cleansing of the GI tract with sodium phosphate. Renal function can be monitored by determining the amount of creatinine clearance by a subject. Normal renal function as used in the methods of the instant invention is defined to be creatinine clearance of greater than or equal to 30 mL/minute.

In another embodiment the instant invention provides methods of determining if a subject is a candidate for cleansing the GI tract with sodium phosphate by determining the subject's age, determining renal function, and/or phosphate level in the kidney. If a subject is determined to be of increased age, they are not a candidate. If a subject is determined to have abnormal levels of phosphate in the kidney they are not a candidate. Finally, if a subject has renal function characterized by creatinine clearance of greater than or equal to 30 mL/minute they are a candidate.

In exemplary methods of the invention once a subject is determined to be a candidate for cleansing the GI tract with sodium phosphate, the subject is administered about 48 mg of sodium phosphate with about 2 L of aqueous solution.

One of skill in the art will recognize that the appropriate dosage of the colonic purgative compositions may vary depending on the individual being treated and the purpose. For example, the age, body weight, and medical history of the individual patient may affect the therapeutic efficacy of the therapy. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Dosages also depend on the strength of the particular purgative(s) chosen for the formulation.

The dose of the colonic purgative formulations may vary. Additional doses of the colonic purgative formulation may be necessary to produce the desired therapeutic effect.

The colonic purgative formulations of the invention may be manufactured in a variety of ways. In one embodiment of the invention, the formulations may be produced using a direct compression or hot-melt process. In an additional embodiment of the invention, a process of producing a colonic purgative formulation comprises mixing the components, warming the mixture to the melting point of the polyethylene glycol, and compressing the mixture into tablets.

In the hot melt process, for instance, the ingredients may be mixed in a high-shear mixer equipped with a jacketed mixing bowl. The blend may be warmed up to the melting point of PEG during mixing and cooling down when the end-point is reached. The blend may be cooled down overnight, milled, lubricated, and compressed into tablets. One of ordinary skill in the art will recognize methods of varying the manufacturing process to optimize the dosage form or increase the product amount for large scale manufacturing.

In certain embodiments, it may be advantageous to co-administer other therapeutics with the GI cleanser. Such co-administered therapeutics include, for example, one or more of an anti-inflammatory, one or more additional antibiotics, an anti emetic, an anti-diarrheal, (e.g., crofelemer or rifaximin), or metoclopramide.

In certain embodiments, other therapeutic agents may be co-administered with the GI cleanser or with the antibiotic or both. These other therapeutic agent(s) may also be given prior to the GI cleanser, during the GI cleanser or between administration of the GI cleanser and the antibiotic.

Particularly suitable antibiotics for use in the methods described herein include, for example neomycin, metronidazole, teicoplanin, doxycycline, tetracycline, ciprofloxacin, augmentin, cephalexin (e.g., Keflex), penicillin, ampicillin, kanamycin, rifamycin, rifaximin or vancomycin, which may be administered orally, intravenously, rectally or other method found useful by one of skill in the art, such as through a feeding tube or stoma. (R. K. Cleary [1998]; C. P. Kelly and J. T. LaMont, Clostridium difficile infection, Annu. Rev. Med. 49′375-90 [1998]; C. M. Reinke and C. R Messick, Update on Clostridium difficile-induced colitis, Part 2, Am. J. Hosp. Pharm. 51(15):1892-1901 [1994]).

In certain embodiments, it is advantageous to administer an anti-inflammatory composition.

### Pharmaceutical Preparations

The phrase “pharmaceutically acceptable” refers to those antibiotics and GI cleansers described herein, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an antibiotic(s) as an active ingredient. A compound may also be administered as a bolus, electuary or paste.

Embodiments relate to all of the solid preparations administrable by the oral route, for instance coated and uncoated tablets, of soft and hard gelatin capsules, sugar-coated pills, lozenges, wafer sheets, pellets and powders in sealed packets or other containers.

In solid dosage forms for oral administration (capsules, tablets, pills, dragees, powders, granules and the like), the active ingredient is typically mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) colouring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the sodium phosphate GI cleanser formulation, such as dragees, capsules, pills and granules, may optionally be scored or prepared with coatings and shells, such as enteric coatings and other coatings well known in the pharmaceutical-formulating art.

They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral or rectal administration of the antibiotic(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Actual dosage levels and time course of administration of the active ingredients in the pharmaceutical compositions may be varied so as to obtain an amount of the active ingredient which is effective to achieve the desired therapeutic response for a particular patient, composition, and mode of administration, without being toxic to the patient.

In combination therapy treatment, both the compounds and the other drug agent(s) are administered to subjects (e.g., humans, male or female) by appropriate methods. The agents may be administered in a single dosage form or in separate dosage forms. Effective amounts of the other therapeutic agents for particular purposes are well known to those skilled in the art. However, it is well within the skilled artisan's purview to determine the other therapeutic agent's optimal effective-amount range. In one embodiment in which another therapeutic agent is administered to a subject, the effective amount of the compound is less than its effective amount in case the other therapeutic agent is not administered. In another embodiment, the effective amount of the agent is less than its effective amount in case the compound is not administered. In this way, undesired side effects associated with high doses of either agent may be minimized. Other potential advantages (including without limitation improved dosing regimens and/or reduced drug cost) will be apparent to those skilled in the art.

In certain embodiments, the administration of the same compounds may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an antibiotic may be repeated and the administration may be separated by at least at least 1 day, 2 days, 3 days, 5 days, 10 days, 15 days, 30 days, 45 days, 2 months, 75 days, 3 months, or at least 6 months.

### Article of Manufacture

Another embodiment includes articles of manufacture that comprise, for example, a container holding a GI cleanser pharmaceutical composition with printed labeling instructions providing a discussion of when a particular composition and dosage form should be administered. Exemplary dosage forms and administration protocols are described infra. The composition will be contained in any suitable container capable of holding and dispensing the dosage form and which will not significantly interact with the composition and will further be in physical relation with the appropriate labeling. The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

Another aspect is an article of manufacture that comprises a container containing a pharmaceutical composition comprising a GI cleanser wherein the container holds a GI cleanser in ready to drink or administer formulation and is associated with printed labeling instructions advising the subject how to take the composition.

Packaged compositions are also provided, and may comprise a therapeutically effective amount of a GI cleanser.

Kits are also provided herein, for example, kits for cleansing the colon prior to a colonoscopy or abdominal surgery. The kits may contain, for example, a GI cleanser and instructions for use. The kits may also contain a medication guide. The instructions for use may contain proscribing information, dosage information, storage information, and the like. Medication guides, may for example, answer questions that my occur to a subject regarding the methods of taking the cleanser or the risks associated with taking the GI cleanser.

Instructions for OsmoPrep may include, for example, one or more of the following sections in a prominent position on the instruction sheet, for example, it may be contained within a box:
WARNINGS
   There have been rare, but serious reports of acute phosphate nephropathy in patients who received oral sodium phosphate products for colon cleansing prior to colonoscopy. Some cases have resulted in permanent impairment of renal function and some patients required long-term dialysis. While some cases have occurred in patients without identifiable risk factors, patients at increased risk of acute phosphate nephropathy may include those with increased age, hypovolemia, increased bowel transit time (such as bowel obstruction), active colitis, or baseline kidney disease, and those using medicines that affect renal perfusion or function (such as diuretics, angiotensin converting enzyme [ACE] inhibitors, angiotensin receptor blockers [ARBs], and possibly nonsteriodal anti-inflammatory drugs [NSAIDs]).
   It is important to use the dose and dosing regimen as recommended (pm/am split dose).

Other Sections of the instruction sheet, may contain one or more of the following sections:
DOSAGE and ADMINISTRATION.
   OsmoPrep (sodium phosphate monobasic monohydrate, USP, and sodium phosphate dibasic anhydrous, USP) is a purgative used to clean the colon prior to colonoscopy. OsmoPrep is manufactured with a highly soluble tablet binder and does not contain microcrystalline cellulose (MCC). OsmoPrep Tablets are oval, white to off-white compressed tablets, debossed with “SLX” on one side of the bisect and “102” on the other side of the bisect. Each OsmoPrep tablet contains 1.102 grams of sodium phosphate monobasic monohydrate, USP and 0.398 grams of sodium phosphate dibasic anhydrous, USP for a total of 1.5 grams of sodium phosphate per tablet. Inert ingredients include polyethylene glycol 8000, NF; and magnesium stearate, NF. OsmoPrep is gluten-free.

Sodium phosphate monobasic monohydrate, USP

Molecular Formula NaH2PO4.H2O

Molecular Weight: 137.99

Sodium phosphate dibasic anhydrous, USP

Molecular Formula Na2HPO4

Molecular Weight: 141.96
OsmoPrep Tablets are for oral administration only.
CLINICAL PHARMACOLOGY
   OsmoPrep Tablets, a dosing regimen containing 48 grams of sodium phosphate (32 tablets), induces diarrhea, which effectively cleanses the entire colon. Each administration has a purgative effect for approximately 1 to 3 hours. The primary mode of action is thought to be through the osmotic effect of sodium, causing large amounts of water to be drawn into the colon, promoting evacuation.
Pharmacokinetics
   Pharmacokinetic studies with OsmoPrep have not been conducted. However, the following pharmacokinetic study was conducted with Visicol tablets which contain the same active ingredients (sodium phosphate) as OsmoPrep. In addition, Visicol is administered at a dose that is 25% greater than the OsmoPrep dose.
   An open-label pharmacokinetic study of Visicol in healthy volunteers was performed to determine the concentration-time profile of serum inorganic phosphorus levels after Visicol administration. All subjects received the approved Visicol dosing regimen (60 grams of sodium phosphate with a total liquid volume of 3.6 quarts) for colon cleansing. A 30 gram dose (20 tablets given as 3 tablets every 15 minutes with 8 ounces of clear liquids) was given beginning at 6 PM in the evening. The 30 gram dose (20 tablets given as 3 tablets every 15 minutes with 8 ounces of clear liquids) was repeated the following morning beginning at 6 AM.
   Twenty-three healthy subjects (mean age 57 years old; 57% male and 43% female; and 65% Hispanic, 30% Caucasian, and 4% African-American) participated in this pharmacokinetic study. The serum phosphorus level rose from a mean (±standard deviation) baseline of 4.0 (±0.7) mg/dL to 7.7 (±1.6 mg/dL), at a median of 3 hours after the administration of the first 30 gram dose of sodium phosphate tablets. The serum phosphorus level rose to a mean of 8.4 (±1.9) mg/dL, at a median of 4 hours after the administration of the second 30 gram dose of sodium phosphate tablets. The serum phosphorus level remained above baseline for a median of 24 hours after the administration of the initial dose of sodium phosphate tablets (range 16 to 48 hours).
Special Populations
   Renal Insufficiency: The effect of renal dysfunction on the pharmacokinetics of OsmoPrep Tablets has not been studied. Since the inorganic form of phosphate in the circulating plasma is excreted almost entirely by the kidneys, patients with renal disease may have difficulty excreting a large phosphate load. Thus, OsmoPrep Tablets should be used with caution in patients with impaired renal function (see WARNINGS).
   Hepatic Insufficiency: OsmoPrep Tablets have not been investigated in patients with hepatic failure.
   Geriatric: In a single pharmacokinetic study of sodium phosphate tablets, which included 6 elderly volunteers, plasma half-life increased two-fold in subjects >70 years of age compared to subjects <50 years of age (3 subjects and 5 subjects, respectively).
   Gender: No difference in serum phosphate AUC values were observed in the single pharmacokinetic study conducted with sodium phosphate tablets in 13 male and 10 female healthy volunteers.
CLINICAL STUDIES
   The colon cleansing efficacy and safety of OsmoPrep was evaluated in 2 randomized, investigator-blinded, actively controlled, multicenter, U.S. trials in patients scheduled to have an elective colonoscopy. The trials consisted of a dose ranging and a confirmatory phase 3 study.
   In the phase 3 trial, patients were randomized into one of the following three sodium phosphate treatment groups: 1) Visicol containing 60 grams of sodium phosphate given in split doses (30 grams in the evening before the colonoscopy and 30 grams on the next day) with at least 3.6 quarts of clear liquids; 2) OsmoPrep containing 60 grams of sodium phosphate given in split doses (30 grams in the evening before the colonoscopy and 30 grams on the next day) with 2.5 quarts of clear liquids; and 3) OsmoPrep containing 48 grams of sodium phosphate (30 grams in the evening before the colonoscopy and 18 grams on the next day) with 2 quarts of clear liquids. Patients were instructed to eat a light breakfast before noon on the day prior to the colonoscopy and then were told to drink only clear liquids after noon on the day prior to the colonoscopy.
   The primary efficacy endpoint was the overall colon cleansing response rate in the 4-point Colonic Contents Scale. Response was defined as a rating of “excellent” or “good” on the 4point scale as determined by the blinded colonoscopist. This phase 3 study was planned to assess the non-inferiority of the two OsmoPrep groups compared to the Visicol group.
   The efficacy analysis included 704 adult patients who had an elective colonoscopy. Patients ranged in age from 21 to 89 years old (mean age 56 years old) with 55% female and 45% male patients. Race was distributed as follows: 87% Caucasian, 10% African American, and 3% other race. The OsmoPrep 60 gram and 48 gram treatment groups demonstrated non-inferiority compared to Visicol.
Electrolyte Changes
   In the OsmoPrep clinical studies, expected serum electrolyte changes (including phosphate, calcium, potassium, and sodium levels) have been observed in patients taking OsmoPrep. In the overwhelming majority of patients, electrolyte abnormalities were not associated with any adverse events.
   In the OsmoPrep phase 3 study, 96%, 96%, and 93% of patients who took 60 grams of Visicol, 60 grams of OsmoPrep, and 48 grams of OsmoPrep, respectively, developed hyperphosphatemia (defined as phosphate level >5.1 mg/dL) on the day of the colonoscopy. In this study, patients who took 60 grams of Visicol, 60 grams of OsmoPrep, and 48 grams of OsmoPrep had baseline mean phosphate levels of 3.5, 3.5, and 3.6 mg/dL and subsequently developed mean phosphate levels of 7.6, 7.9, and 7.1 mg/dL, respectively, on the day of the colonoscopy.
   In the OsmoPrep phase 3 study, 20%, 22%, and 18% of patients who took 60 grams of Visicol, 60 grams of OsmoPrep, and 48 grams of OsmoPrep, respectively, developed hypokalemia (defined as a potassium level <3.4 mEq/L) on the day of the colonoscopy. In this study, patients who took 60 grams of Visicol, 60 grams of OsmoPrep, and 48 grams of OsmoPrep all had baseline potassium levels of about 4.3 mEq/L and then developed a mean potassium level of 3.7 mEq/L on the day of the colonoscopy.
   In the OsmoPrep phase 3 trial, several patients on all three sodium phosphate regimens developed hypocalcemia and hypernatremia that did not require treatment.
INDICATIONS AND USAGE
   OsmoPrep Tablets are indicated for cleansing of the colon as a preparation for colonoscopy in adults 18 years of age or older.
CONTRAINDICATIONS
   OsmoPrep Tablets are contraindicated in patients with biopsy-proven acute phosphate nephropathy.
   OsmoPrep Tablets are contraindicated in patients with a known allergy or hypersensitivity to sodium phosphate salts or any of its ingredients.
WARNINGS
   Administration of sodium phosphate products prior to colonoscopy for colon cleansing has resulted in fatalities due to significant fluid shifts, severe electrolyte abnormalities, and cardiac arrhythmias. These fatalities have been observed in patients with renal insufficiency, in patients with bowel perforation, and in patients who misused or overdosed sodium phosphate products. It is recommended that patients receiving OsmoPrep be advised to adequately hydrate before, during, and after the use of OsmoPrep.
   Considerable caution should be advised before OsmoPrep Tablets are used in patients with the following illnesses: severe renal insufficiency (creatinine clearance less than 30 mL/minute), congestive heart failure, ascites, unstable angina, gastric retention, ileus, acute bowel obstruction, pseudo-obstruction of the bowel, severe chronic constipation, bowel perforation, acute colitis, toxic megacolon, gastric bypass or stapling surgery, or hypomotility syndrome.
   Consider performing baseline and post-colonoscopy labs (phosphate, calcium, potassium, sodium, creatinine, and BUN) in patients who may be at increased risk for serious adverse events, including those with history of renal insufficiency, history of—or at greater risk of—acute phosphate nephropathy, known or suspected electrolyte disorders, seizures, arrhythmias, cardiomyopathy, prolonged QT, recent history of a MI and those with known or suspected hyperphosphatemia, hypocalcemia, hypokalemia, and hypernatremia. Also if patients develop vomiting and/or signs of dehydration then measure post-colonoscopy labs (phosphate, calcium, potassium, sodium, creatinine, and BUN).
   Renal Disease, Acute Phosphate Nephropathy, and Electrolyte Disorders
      There have been rare, but serious, reports of renal failure, acute phosphate nephropathy, and nephrocalcinosis in patients who received oral sodium phosphate products (including oral sodium phosphate solutions and tablets) for colon cleansing prior to colonoscopy. These cases often resulted in permanent impairment of renal function and several patients required long-term dialysis. The time to onset is typically within days; however, in some cases, the diagnosis of these events has been delayed up to several months after the ingestion of these products. Patients at increased risk of acute phosphate nephropathy may include patients with the following: hypovolemia, baseline kidney disease, increased age, and patients using medicines that affect renal perfusion or function [such as diuretics, angiotensin converting enzyme (ACE) inhibitors, angiotensin receptor blockers, and possibly nonsteroidal anti-inflammatory drugs (NSAIDs).
   Use OsmoPrep with caution in patients with impaired renal function, patients with a history of acute phosphate nephropathy, known or suspected electrolyte disturbances (such as dehydration), or people taking concomitant medications that may affect electrolyte levels (such as diuretics). Patients with electrolyte abnormalities such as hypernatremia, hyperphosphatemia, hypokalemia, or hypocalcemia should have their electrolytes corrected before treatment with OsmoPrep Tablets.
Seizures
   There have been rare reports of generalized tonic-clonic seizures and/or loss of consciousness associated with use of sodium phosphate products in patients with no prior history of seizures. The seizure cases were associated with electrolyte abnormalities (e.g., hyponatremia, hypokalemia, hypocalcemia, and hypomagnesemia) and low serum osmolality. The neurologic abnormalities resolved with correction of fluid and electrolyte abnormalities. OsmoPrep should be used with caution in patients with a history of seizures and in patients at higher risk of seizure [patients using concomitant medications that lower the seizure threshold (such as tricyclic antidepressants), patients withdrawing from alcohol or benzodiazepines, or patients with known or suspected hyponatremia].
Cardiac Arrhythmias
   There have been rare, but serious, reports of arrhythmias associated with the use of sodium phosphate products. OsmoPrep should be used with caution in patients with higher risk of arrhythmias (patients with a history of cardiomyopathy, patients with prolonged QT, patients with a history of uncontrolled arrhythmias, and patients with a recent history of a myocardial infarction). Pre-dose and post-colonoscopy ECGs should be considered in patients with high risk of serious, cardiac arrhythmias.
PRECAUTIONS
   Patients should be instructed to drink 8 ounces of clear liquids with each 4-tablet dose of OsmoPrep Tablets. Patients should take a total of 2 quarts of clear liquids with OsmoPrep. Inadequate fluid intake, as with any effective purgative, may lead to excessive fluid loss, hypovolemia, and dehydration. Dehydration from purgation may be exacerbated by inadequate oral fluid intake, vomiting, and/or use of diuretics. Patients should be instructed not to administer additional laxative or purgative agents, particularly additional sodium phosphate-based purgative or enema products. Prolongation of the QT interval has been observed in some patients who were dosed with sodium phosphate colon preparations. QT prolongation with sodium phosphate tablets has been associated with electrolyte imbalances, such as hypokalemia and hypocalcemia. OsmoPrep Tablets should be used with caution in patients who are taking medications known to prolong the QT interval, since serious complications may occur. Pre-dose and post-colonoscopy ECGs should be considered in patients with known prolonged QT.
   Administration of OsmoPrep Tablets may induce colonic mucosal aphthous ulcerations, since this endoscopic finding was observed with other sodium phosphate cathartic preparations. In the OsmoPrep clinical program, aphthous ulcers were observed in 3% of patients who took the 48 gram OsmoPrep dosing regimen. This colonoscopic finding should be considered in patients with known or suspected inflammatory bowel disease. Because published data suggest that sodium phosphate absorption may be enhanced in patients experiencing an acute exacerbation of chronic inflammatory bowel disease, OsmoPrep Tablets should be used with caution in such patients.
Drug Interactions
   Medications administered in close proximity to OsmoPrep Tablets may not be absorbed from the gastrointestinal tract due to the rapid intestinal peristalsis and watery diarrhea induced by the purgative agent.
Carcinogenesis, Mutagenesis, Impairment of Fertility
   Long-term studies in animals have not been performed to evaluate the carcinogenic potential of OsmoPrep. Studies to evaluate the effect of OsmoPrep on fertility or its mutagenic potential have not been performed.
Pregnancy. Teratogenic Effects: Pregnancy Category C
   Animal reproduction studies have not been conducted with OsmoPrep. It is not known whether OsmoPrep can cause fetal harm when administered to a pregnant woman, or can affect reproduction capacity. OsmoPrep Tablets should be given to a pregnant woman only if clearly needed.
Pediatric Use
   The safety and efficacy of OsmoPrep Tablets have not been demonstrated in patients less than 18 years of age.
Geriatric Use
   In controlled colon preparation trials of OsmoPrep, 228 (24%) of 931 patients were 65 years of age or older. In addition, 49 (5%) of the 931 patients were 75 years of age or older.
   Of the 228 geriatric patients in the trials, 134 patients (59%) received at least 48 grams of OsmoPrep. Of the 49 patients 75 years old or older in the trials, 27 (55%) patients received at least 48 grams of OsmoPrep. No overall differences in safety or effectiveness were observed between geriatric patients and younger patients. However, the mean phosphate levels in geriatric patients were greater than the phosphate levels in younger patients after OsmoPrep administration. The mean colonoscopy-day phosphate levels in patients 18-64, 65-74, and =75 years old who received 48 grams of OsmoPrep in the phase 3 study were 7.0, 7.3, and 8.0 mg/dL, respectively. In addition, in all three sodium phosphate treatment groups, the mean phosphate levels in patients 18-64, 65-74, and =75 years old in the phase 3 study were 7.4, 7.9, and 8.0 mg/dL, respectively, after sodium phosphate administration. Greater sensitivity of some older individuals cannot be ruled out; therefore, OsmoPrep Tablets should be used with caution in geriatric patients.
   Sodium phosphate is known to be substantially excreted by the kidney, and the risk of adverse reactions with sodium phosphate may be greater in patients with impaired renal function. Since geriatric patients are more likely to have impaired renal function, consider performing baseline and post-colonoscopy labs (phosphate, calcium, potassium, sodium, creatinine, and BUN) in these patients (see WARNINGS). It is recommended that patients receiving OsmoPrep be advised to adequately hydrate before, during, and after the use of OsmoPrep.
ADVERSE REACTIONS
   Abdominal bloating, abdominal pain, nausea, and vomiting were the most common adverse events reported with the use of OsmoPrep Tablets. Dizziness and headache were reported less frequently. Since diarrhea was considered as a part of the efficacy of OsmoPrep, diarrhea was not defined as an adverse event in the clinical studies. The most common adverse events associated with the use of 48 grams of OsmoPrep, 60 grams of OsmoPrep, and 60 grams of Visicol in the colon preparation trials (n=931) are listed below.
   Frequency of Adverse Events of Any Severity Occurring in Greater Than 3% of Patients in the OsmoPrep Trials

OsmoPrep 32 tabs (48 g) N=272; OsmoPrep 40 tabs (60 g) N=265; Visicol 40 tabs (60 g) N=268

Bloating 31% 39% 41%

Nausea 26% 37% 30%

Abdominal Pain 23% 24% 25%

Vomiting 4% 10% 9%
Postmarketing Experience
   In addition to adverse events reported from clinical trials, the following adverse events have been identified during post-approval use of OsmoPrep. Because they are reported voluntarily from a population of unknown size, estimates of frequency cannot be made. These events have been chosen for inclusion due to either their seriousness, frequency of reporting or causal connection to OsmoPrep, or a combination of these factors.
   General: Hypersensitivity reactions including anaphylaxis, rash, pruritus, urticaria, throat tightness, bronchospasm, dyspnea, pharyngeal edema, dysphagia, paresthesia and swelling of the lips and tongue, and facial swelling. Cardiovascular: Arrhythmias; Nervous system: Seizures; and Renal: Renal impairment, increased blood urea nitrogen (BUN), increased creatinine, acute renal failure, acute phosphate nephropathy, nephrocalcinosis, and renal tubular necrosis.
DRUG ABUSE AND DEPENDENCE
   Laxatives and purgatives (including OsmoPrep) have the potential for abuse by bulimia nervosa patients who frequently have binge eating and vomiting.
OVERDOSAGE
   There have been no reported cases of overdosage with OsmoPrep Tablets. Purposeful or accidental ingestion of more than the recommended dosage of OsmoPrep Tablets might be expected to lead to severe electrolyte disturbances, including hyperphosphatemia, hypocalcemia, hypernatremia, or hypokalemia, as well as dehydration and hypovolemia, with attendant signs and symptoms of these disturbances. Certain severe electrolyte disturbances resulting from overdose may lead to cardiac arrhythmias, seizure, renal failure, and death. The patient who has taken an overdosage should be monitored carefully, and treated symptomatically for complications until stable.
DOSAGE AND ADMINISTRATION
   The recommended dose of OsmoPrep Tablets for colon cleansing for adult patients is 32 tablets (48 grams of sodium phosphate) taken orally with a total of 2 quarts of clear liquids in the following manner:

The evening before the colonoscopy procedure: Take 4 OsmoPrep Tablets with 8 ounces of clear liquids every 15 minutes for a total of 20 tablets.

On the day of the colonoscopy procedure: Starting 3-5 hours before the procedure, take 4 OsmoPrep Tablets with 8 ounces of clear liquids every 15 minutes for a total of 12 tablets.
Patients should be advised of the importance of taking the recommended fluid regimen. It is recommended that patients receiving OsmoPrep be advised to adequately hydrate before, during, and after the use of OsmoPrep.
Patients should not use OsmoPrep for colon cleansing within seven days of previous administration. No additional enema or laxative is required, and patients should be advised NOT to take additional agents, particularly those containing sodium phosphate.
   HOW SUPPLIED
OsmoPrep Tablets are supplied in child-resistant bottles containing 100 tablets.

Each tablet contains 1.102 g sodium phosphate monobasic monohydrate, USP and 0.398 g sodium phosphate dibasic anhydrous, USP for a total of 1.5 g of sodium phosphate per tablet.
Each bottle contains two silica desiccant packets, which should not be ingested. In certain embodiments, a medication guide is included in a kit. Medication guides may include one or more of the following sections. The following guide, as an example, uses the OsmoPrep product, but this type of guide may be used for any sodium phosphate purgative products.
   Read the Medication Guide that comes with OsmoPrep before you take it and each time you take it. This Medication Guide does not take the place of talking with your doctor about your medical condition or your treatment. If you have any questions about OsmoPrep, ask your doctor or pharmacist.
   What is the most important information I should know about OsmoPrep?
OsmoPrep can cause serious side effects, including:
   Serious kidney problems. Rare, but serious kidney problems can happen in people who take medicines made with sodium phosphate, including OsmoPrep, to clean your colon before a colonoscopy. These kidney problems can sometimes lead to kidney failure or the need for dialysis for a long time. These problems often happen within a few days, but sometimes may happen several months after taking OsmoPrep.
Conditions that can make you more at risk for having serious kidney problems with

OsmoPrep include if you: lose too much body fluid (dehydration); have slow moving bowels; have bowels blocked with stool (constipation); have severe stomach pain or bloating; have any disease that causes bowel irritation (colitis); have kidney disease; have heart failure; take water pills or non-steroidal anti-inflammatory drugs (NSAIDS).
Your age may also affect your risk for having kidney problems with OsmoPrep.
Before you start taking OsmoPrep, tell your doctor if you: have kidney problems;
   take any medicines for blood pressure, heart disease, or kidney disease. Severe fluid loss. People who take medicines that contain sodium phosphate can have severe loss of body fluid, with severe changes in body salts in the blood, and abnormal heart rhythms. These problems can lead to death.
Tell your doctor if you have any of these symptoms of loss of too much body fluid (dehydration) while taking OsmoPrep: vomiting; dizziness; urinating less often than normal; headache.
   What is OsmoPrep?
OsmoPrep is a prescription medicine used in adults 18 years and older, to clean your colon before a colonoscopy. OsmoPrep cleans your colon by causing you to have diarrhea. Cleaning your colon helps your doctor see the inside of your colon more clearly during the colonoscopy.
   It is not known if OsmoPrep is safe and works in children under age 18.
   Who should not take OsmoPrep?
Do not take OsmoPrep if: you have had a kidney biopsy that shows you have kidney problems because of too much phosphate; you are allergic to sodium phosphate salts or any of the ingredients in OsmoPrep.
   What should I tell my doctor before taking OsmoPrep?
Before taking OsmoPrep, tell your doctor about all your medical conditions, including if you have: any of the medical conditions listed in the section “What is the most important information I should know about OsmoPrep?”; irritation of the bowel (colitis). OsmoPrep can cause symptoms of irritable bowel disease to flare-up; damage to your bowels; problems with abnormal heart beat; had a recent heart attack or have other heart problems; symptoms of too much body fluid loss (dehydration) including vomiting, dizziness, urinating less often than normal, or headache; had stomach surgery; a history of seizures; if you drink alcohol; are on a low salt diet; are pregnant. It is not known if OsmoPrep will harm your unborn baby.
Tell your doctor about all the medicines you take, including prescription and nonprescription medicines, vitamins, and herbal supplements. Any medicine that you take close to the time that you take OsmoPrep may not work as well. Especially tell your doctor if you take: water pills (diuretics); medicines for blood pressure or heart problems; medicines for kidney damage; medicines for pain, such as aspirin or a non-steroidal anti-inflammatory drug (NSAID); a medicine for seizures and/or a laxative for constipation in the last 7 days.
You should not take another medicine that contains sodium phosphate while you take OsmoPrep.
Ask your doctor if you are not sure if your medicine is listed above. Know the medicines you take. Keep a list of your medicines to show your doctor or pharmacist when you get a new prescription.
   How should I take OsmoPrep? Take OsmoPrep exactly as prescribed by your doctor. It is important for you to drink clear liquids before, during, and after taking OsmoPrep. This may help prevent kidney damage. Examples of clear liquids are water, flavored water, lemonade (no pulp), ginger ale, or apple juice. Do not drink any liquids colored purple or red. You must read, understand, and follow these instructions to take OsmoPrep the right way:
      On the evening before your colonoscopy, you will take a total of 20 OsmoPrep tablets, as follows:
      1. Take 4 OsmoPrep tablets with 8 ounces of clear liquids.
      2. Wait 15 minutes.
      3. Take 4 more OsmoPrep tablets with 8 ounces of clear liquids.
      4. Repeat steps 2 and 3 above, three more times. Make sure you wait 15 minutes after each time.
      On the day of your colonoscopy you will take a total of 12 OsmoPrep tablets, starting about 3 to 5 hours before your colonoscopy, as follows:
      1. Take 4 OsmoPrep tablets with 8 ounces of clear liquids.
      2. Wait 15 minutes.
      3. Take 4 more OsmoPrep tablets with 8 ounces of clear liquids.
      4. Repeat steps 2 and 3 one more time.
Tell your doctor if you have any of these symptoms while taking OsmoPrep: vomiting, dizziness, or if you urinate less often than normal. These may be signs that you have lost too much fluid while taking OsmoPrep; trouble drinking clear fluids; severe stomach cramping, bloating, nausea, or headache.
If you take too much OsmoPrep, call your doctor or get medical help right away.
What should I avoid while taking OsmoPrep? You should not take other laxatives or enemas made with sodium phosphate, while taking OsmoPrep.
You should not use OsmoPrep if you have already used it in the last 7 days.
What are the possible side effects of OsmoPrep? OsmoPrep can cause serious side effects, including: Seizures or fainting (black-outs). People who take a medicine that contains sodium phosphate, such as OsmoPrep, can have seizures or faint (become unconscious) even if they have not had seizures before. Tell your doctor right away if you have a seizure or faint while taking OsmoPrep; abnormal heart beat (arrhythmias); changes in your blood levels of calcium, phosphate, potassium, sodium.
The most common side effects of OsmoPrep are: bloating; stomach area (abdominal) pain; nausea; and vomiting. These are not all the possible side effects of OsmoPrep. For more information, ask your doctor or pharmacist.
How do I store OsmoPrep? Store OsmoPrep at room temperature, between 59° F. to 86° F. (15° C. to 30° C.). Throw away any OsmoPrep that is not needed. Keep OsmoPrep and all medicines out of the reach of children.
General information about OsmoPrep
Medicines are sometimes prescribed for purposes other than those listed in a Medication Guide. Do not use OsmoPrep for a condition for which it was not prescribed. Do not give OsmoPrep to other people, even if they have the same symptoms that you have. It may harm them.
This Medication Guide summarizes the most important information about OsmoPrep. If you would like more information about OsmoPrep, talk with your doctor or pharmacist. You can ask your doctor or pharmacist for information that is written for healthcare professionals.
What are the ingredients in OsmoPrep? Active ingredients: sodium phosphate monobasic and sodium phosphate dibasic anhydrous Inactive ingredients: polyethylene glycol 8000 and magnesium stearate.

What is claimed is:

1. A method of cleansing the GI tract of a subject, comprising:
administering about 48 mg of sodium phosphate with about 2 L of aqueous solution to a subject who has not recently had a kidney biopsy indicating kidney damage due to excess phosphate.

2. A method of cleansing the GI tract of a subject, comprising:
determining weather a subject is a candidate for a sodium phosphate GI cleanser;
providing about 48 mg of sodium phosphate to a subject; and
instructing the subject to ingest the sodium phosphate with 2 L of aqueous solution.

3. The method of claim 2, wherein determining comprises a biopsy of the kidney, wherein if the biopsy shows damage to the kidney due to too much phosphate, the subject is not a candidate for a sodium phosphate GI cleanser.

4. A method of determining if a subject is a candidate for cleansing the GI tract with sodium phosphate comprising:
determining the phosphate levels in a kidney of the subject;
wherein normal levels of phosphate in the kidney indicates that the subject is a candidate for cleansing of the GI tract with sodium phosphate.

5. The method of claim 4, wherein a biopsy is performed to obtain a sample of the kidney from the subject.

6. The method of claim 4, further comprising administering to a subject determined to be a candidate about 48 mg of sodium phosphate with about 2 L of aqueous solution.

7. A method of determining if a subject is a candidate for cleansing the GI tract with sodium phosphate comprising:
determining if the subject has normal renal function;
wherein normal renal function indicates that the subject is a candidate for cleansing of the GI tract with sodium phosphate.

8. The method of claim 7, wherein normal renal function is characterized by creatinine clearance of greater than or equal to 30 mL/minute.

9. The method of claim 8, wherein further comprising administering to a subject determined to be a candidate about 48 mg of sodium phosphate with about 2 L of aqueous solution.

10. A method of determining if a subject is a candidate for cleansing the GI tract with sodium phosphate comprising:
determining if the subject is a candidate for sodium phosphate purgative cleansing by one or more of: determining age, determining renal function, or determining phosphate level of the kidney; and
administering to a subject determined to be a candidate about 48 mg of sodium phosphate with about 2 L of aqueous solution.

11. The method of claim 10, wherein if a subject is determined to be of increased age, they are not a candidate.

12. The method of claim 10, wherein determined to have abnormal levels of phosphate in the kidney they are not a candidate.

13. The method of claim 10, if a subject has renal function characterized by creatinine clearance of greater than or equal to 30 mL/minute they are a candidate.
